(19) United States
(12) Patent Application Publication
(10) Pub. No.: US 2004/0247582 A1
(43) Pub. Date: Dec. 9, 2004

(54) Feed additive and/or drinking water additive for domestic animals

(76) Inventors: Johann Binder, Tulln (AT); Eva-Maria Binder, Tulln (AT); Sabine Nitsch, St. Polten (AT); Alfred Klimitsch, Reidling (AT)

Correspondence Address: JACOBSON HOLMAN PLLC, 400 SEVENTH STREET N.W., SUITE 600, WASHINGTON, DC, 20004, US

(21) Appl. No.: 10/887,828

(22) Filed: Jul. 12, 2004

Related U.S. Application Data
(63) Continuation of application No. PCT/AT03/00308, filed on Oct. 10, 2003.

(30) Foreign Application Priority Data
Oct. 11, 2002 (AT) A 1543/2002

(51) Int. Cl. A61K 45/00; A61K 31/715

(52) U.S. Cl. 424/93.45; 514/54

(57) ABSTRACT

In a feed additive and/or drinking water additive for domestic animals, which contains at least bacteria of the strain Streptococcus faecium IMB 52 (DSM 3530), it is provided that standardized cell wall components from Bacillus sp. and/or Streptoccocus sp. and/or Bifidobacterium sp. as well as inulin are additionally contained.

The present application is a continuation application of PCT/AT03/00308, filed on Oct. 10, 2003.

### FIELD OF THE INVENTION

The present invention relates to a feed additive and/or drinking water additive for domestic animals, which contains at least bacteria of the strain Streptococcus faecium IMB 52 (DSM 3530).

### DESCRIPTION OF THE PRIOR ART

The use of bacteria as probiotic components in feed and/or drinking water additives for domestic animals has been known for long, for instance from AT-B 383 856. In that AT-B 383 856, strains from the Enterococcus group and, in particular, the strain Streptococcus faecium IMB 52 (DSM 3530) are admixed to the feed to function as microbial growth promoters. Growth promoters in this respect relate to feed additives that encourage the productivity of animals for slaughter, particularly in respect to meat production, based on the feed amount and feeding time employed. In addition, it could be demonstrated that the colonization of pathogenic bacteria and/or viruses in the intestine could be markedly reduced, as could the formation of toxins by pathogens, when using the strain Streptococcus faecium IMB 52 (DSM 3530).

Feed and drinking water additives aimed to enhance the immunity of livestock or domestic animals have been investigated and tested for quite some time in order to minimize or alleviate special strains exerted on the immune system of the organism particularly during stress phases of domestic animals. Thus, young animals are especially strained by stress when weaning young pigs or during regroupings or transport-related overloads or feed changeovers in the fattening of calves and poultry. Being stress factors too, any chronic disease or any continuing traumatism will also strain the organisms of domestic animals, and hence reduce their productivity. The animal organism is noticeably strained during such stress phases, yet an animal can undergo hidden defence weaknesses at any other point of time, thus being particularly prone to the action of both potentially pathogenic and pathogenic germs as well as other noxae. At that time, factor diseases are particularly likely to occur, leading to direct efficiency depressions in domestic animals, which will finally result in non-negligible economic losses for the livestock breeder.

At present, attempts have focussed on counteracting stress or disease factors occurring in the critical phases of domestic livestock breeding such as, for instance, during the weaning of young pigs or the first days of life of young chicks, by the prophylactic or metaphylactic administration of bactericidal antibiotics. Particularly with a view to eliminating growth-restrained bacteria, a functioning immune system is, however, presupposed in the event of bacteriostatics and chemo therapeutics, which is, of course, not always present in domestic animals subjected to elevated stress phases. The use of antibiotics, in addition to the insecurity as to the physical condition of the treated animal, also involves the disadvantage that waiting times have to be observed after the administration of antibiotics and prior to supplying an animal to food production. These waiting times, which are stipulated by law, relate to the metabolisms of healthy animals, and it goes without saying that the metabolism of a sick animal, which strongly deviates from that of a healthy one, is frequently unable to completely degrade antibiotics even after the indicated waiting times. As a result, an increased number of precise residue checks will have to be carried out in order to ensure that foods derived from the animal will not jeopardize consumers.

Moreover, it must be taken into account that the majority of bacterial strains will become resistant against the administered antibiotics over time and the effects of theses substances will therefore diminish. As a result, the dosage of such antibiotics will have to be increased, which will, in turn, call for extended waiting times. Overall, there is no doubt about the fact that the disadvantages involved in the administration of antibiotics not only reside in problems of residues and toxicity as well as the resistance of animals, but that, in the long run, the administration of antibiotics should also be avoided in view of the people's health.

It is, furthermore, known from the literature that various carbohydrates and non-starch poly- or oligosaccharides play important roles in dietetics. In this context, it is, for instance, referred to the document entitled “Dietary Modulation of the Human Gut Microflora Using the Prebiotics Oligofructose and Inulin”, Glenn R. Gibson, American Society for Nutritional Sciences, 1999, pp. 1438 et seq., where the use of prebiotics in nutriments is discussed amongst others, and it is pointed out that such substances may provide an enhanced resistance against pathogenic germs and the like.

### SUMMARY OF THE INVENTION

The present invention aims to provide a feed additive and/or drinking water additive which strengthens the innate immune system of livestock or domestic animals and, at the same time, prevents the formation of toxins by pathogens and the colonization of pathogens in the intestines so as to not only encourage the productivity of domestic animals, but also significantly reduce their dropout rates.

To solve this object, the feed additive and/or drinking water additive according to the invention is essentially characterized in that standardized cell wall components from Bacillus sp. and/or Streptoccocus sp. and/or Bifidobacterium sp. as well as inulin are additionally contained. Due to the fact that the feed additive and/or drinking water additive besides Streptococcus faecium IMB 52 (DSM 3530) as a probiotic component also contains standardized cell wall components, namely Bacillus sp. and/or Streptoccocus sp. and/or Bifidobacterium sp., which exhibit, in particular, immunostimulating capacities caused by the interaction with surface receptors of macrophages, an enhanced macrophage activity will be obtained, which will strengthen the innate immune system as a whole. The enhanced macrophage activity, which is due, in particular, to the increased phagocytic activity of the macrophages, causes animals that have been administered standardized cell wall components to exhibit an elevated resistance to infections, and hence an accelerated growth and reduced number of losses. The simultaneous administration of a probiotic component, i.e., Streptococcus faecium IMB 52 (DSM 3530), results in a rapid proliferation within the intestines and hence a colonization of the same, which, in turn, prevents the settlement of pathogens in the intestines. At the same time, Streptococcus faecium IMB 52 (DSM 3530) exhibits an antagonistic action against pathogens such that the combined administration of cell wall components and the probiotic component will markedly raise the resistance against infections on the one hand and simultaneously prevent the colonization of the intestines with pathogens on the other hand, so that the dropout rate of animals can be clearly reduced during breeding. Due to the fact that the feed additive and/or drinking water additive also contains a prebiotic fructooligosaccharide besides the probiotic component and the standardized cell wall components, the growth of bifidobacteria, which are able to utilize prebiotic fructooligosaccharides specifically as dietary sources, and hence the colonization of the intestines of animals with natural gut inhabitants are stimulated, which, in turn, prevents pathogens from settling there. Moreover, the administration of prebiotic fructooligosaccharides may cause a shift in the balance of the intestinal flora towards positive germs and prevent pathogenic germs from migrating upwards into the small bowel particularly on account of the action of the prebiotic fructooligosaccharides over the entire gastro-intestinal tract of the animal. In this manner, it is feasible without administering any antibiotics to hamper the colonization of the intestines with pathogens almost completely.

According to a further development of the invention, the standardized cell wall components are selected from the strain Bacillus subtilis. Cell wall components from the cited strain are relatively simple to produce, or inactivate, by current production methods and can, therefore, be economically produced in larger quantities. Moreover, cell wall components of the cited strain, in particular, exhibit especially good immunostimulating capacities due to the interaction with surface receptors of macrophages, so that they will be used in a preferred manner.

According to a further development, the standardized cell wall components from Bacillus subtilis are contained in an amount of from 0.2 to 40 g/kg feed additive and/or drinking water additive and, in particular, 5 to 15 g/kg. Screenings demonstrated that it was feasible through the use of Bacillus subtilis as standardized cell wall components in amounts of from 0.2 to 40 g/kg feed additive and/or drinking water additive, along with the use of the strain Streptococcus faecium IMB 52 (DSM 3530) in amounts of from 1×10⁷ to 1×10¹⁷ cfu/kg, to obtain a synergistic effect between the probiotic component and the cell wall components, which effect largely exceeded the cumulative effect of the individual components, thus largely increasing the activation action on the cells, which can only be explained by the synergism of the probiotic component with Bacillus subtilis.

According to a further development, inulin is isolated from leek, onion, garlic, artichoke, wheat, chicory, topinambour, tomato, banana and/or rye and contained in an amount of from 100 to 950 g/kg feed additive and/or drinking water additive. According to a further development, said inulin is contained in the feed additive in an amount of from 100 to 700 g/kg, particularly 320 to 540 g/kg, and in the drinking water additive in an amount of from 530 to 950 g/kg, particularly 820 to 920 g/kg. Especially when using drinking water additives, inulin is of particular importance as a prebiotically acting fructooligosaccharide, since it can be employed in large amounts, being readily water-soluble, and hence induce an extremely good stimulation of the growth of bifidobacteria in the gastrointestinal tract of animals. When using inulin in feed additives, smaller amounts may be employed, since, in particular, as in correspondence with a further development of the present invention, brown marine algae are additionally contained in the feed additive and/or drinking water additive as a phycophytic component.

According to a further development, said brown marine algae are selected from Ascophyllum nodosum. Brown marine algae contained as phycophytic components in a feed additive and/or drinking water additive behave in the same manner as cell wall components, yet can be used in considerably larger amounts because of their easier accessibility and extraction as opposed to cell wall components. In accordance with the invention, said brown marine algae according to a further development are contained in an amount of from 300 to 800 g/kg, particularly 450 to 550 g/kg, feed additive. Brown marine algae exhibit a relatively low water solubility such that their use in drinking water additives is feasible only to a limited extent, for which reason the elevated amounts proposed according to the invention can be employed merely in feed additives. On account of the elevated amounts of phycophytic components employed, the amount of inulin used in feed additives can, therefore, be reduced, since the colonization of pathogens will be safely avoided by the immunostimulating capacity of the phycophytic components. For this reason, no particular attention need be paid to the stimulation of the growth of bifidobacteria in this case, so that markedly smaller amounts of inulin can be used as a probiotic fructooligosaccharide in the feed additive than in the drinking water additive.

Moreover, brown marine algae as phycophytic components, together with the probiotic component, show the same synergistic effect exceeding the cumulative effect of the individual components as do the cell wall components, so that, when using the phycophytic component together with cell wall components, namely Bacillus subtilis, and the probiotic component, namely inulin, both the colonization of pathogens in the gastrointestinal tract of animals and the macrophage activity could be enhanced to the extent that both the loss rate of animals could be reduced and their feed conversion could be markedly increased in view of the sum of the effects of the individual components. This increase will be particularly significant if, as in correspondence with a further development of the invention, 10 to 15% of the bacteria of the strain Streptococcus faecium IMB 52 (DSM 3530) are replaced with at least one further bacterial strain.

According to a further development of the invention, the at least one further bacterial strain is selected from the strains Bifidobacterium thermophilum (I-01) (DSM 14411), Bifidobacterium thermophilum (I-07) (DSM 14412), Bifidobacteruim boum (I-12) (DSM 14413), Bifidobacteruim thermophilum (I-15) (DSM 14414), Bifidobacteruim thermophilum (I-19) (DSM 14415) and Bifidobacteruim thermophilum (I-20) (DSM 14416). The bacteria selected from the above-mentioned strains, together with the strain Streptococcus faecium IMB 52 (DSM 3530) exhibit a synergistic effect on account of the increased exploitation of the bifidogenous effect. Moreover, the ammonium excretion of the animals is reduced by the suppression of ammonium-forming bacteria and the ammonium is utilized by the bifidus bacteria to an increased extent, which helps in further strengthening the immune system. Finally, bacteria of the strain Bificobacterium are able to provide an additional, readily absorbable energy source to the animal via the production of short-chain fatty acids and to offer noticeable advantages, particularly in respect to feed conversion and feed utilization by the animals, also in the production of vitamins as well as in the absorption of minerals on account of a reduced pH and an increased solubility of the minerals in the gastrointestinal juice.

According to a further development of the invention, feed additives and/or drinking water additives containing per kg feed additive and/or drinking water additive, respectively, 8 g Bacillus subtilis, 60.0 g Streptococcus faecium IMB 52 (DSM 3530), 432 g Bifidobacteruim thermophilum, balance Ascophyllum nodosum and, in particular, those containing per kg feed additive and/or drinking water additive, respectively, 10 g Bacillus subtilis, 24.75 Streptococcus faecium IMB 52 (DSM 3530), 10.5 g Bifidobacteruim thermophilum (DSM 14414), balance inulin from chicory, have proved to be particularly suitable for the breeding and keeping of animals.

### SHORT DESCRIPTION OF THE DRAWINGS

In the following, the invention will be explained in more detail by way of exemplary embodiments and with reference to the accompanying drawings. Therein,

FIG. 1 depicts the synergistic action of a drinking water additive comprised of standardized cell wall components and a probiotic component, namely Streptococcus faecium IMB 52 (DSM 3530), as against the effect of each individual component;

FIG. 2 depicts the synergistic action of a feed additive comprised of Bacillus sp. and/or Streptococcus sp. and/or Bifidobacterium sp. standardized cell wall components, a probiotic component and brown marine algae, as against the effect of each individual component;

FIG. 3 is a diagram of a drinking water additive, illustrating the results of the test substances in percent of the phagocytosis/control, as against the effect of each individual component; and

FIG. 4 is a diagram illustrating the results of the test substances in a feed additive in percent of the phagocytosis/control, as against the effect of each individual component.

### DETAILED DESCRIPTION OF PREFERRED EMBODIMENTS

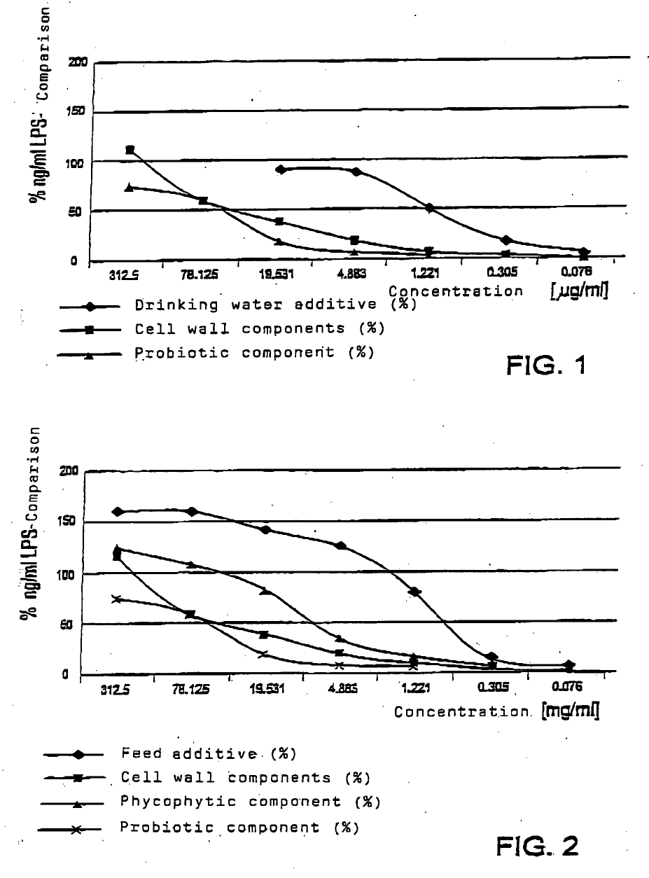

In the diagram of FIG. 1, the results from tests relating to the action of products according to the present invention as opposed to the action of the individual substances are plotted as concentrations against % positive control, and it is apparent that, although the individual substances have stimulating effects on macrophages, the combination exhibits a synergistic effect largely exceeding the cumulative effect of the individual components, particularly at a concentration range of about 0.2 to 19.5 μg/ml. It is, thus, feasible by the aid of a drinking water additive composed of standardized cell wall components and a probiotic component and supplemented with inulin, to extremely stimulate the macrophages present in the gastrointestinal tract and prevent pathogens from colonizing there.

The inulin additionally contained in the drinking water additive as a prebiotic fructooligosaccharide in this case does not contribute anything to the synergistic effect, since it merely stimulates the growth of the bifidobacteria and hence exhibits a bifidogenous effect, which is definitely a desired additional effect, yet irrelevant in respect to macrophage activity and the prevention of pathogen colonization.

FIG. 2 in an analogous diagram shows the synergistic action of a feed additive comprising the phycophytic component in addition to the probiotic component and the standardizied cell wall components. It is clearly apparent from FIG. 2 that the synergistic effect is particularly high in this case, especially at concentrations of about 0.5 to 5 mg/ml, and the use of slight amounts of the feed additive according to the invention, thus, yields extremely good results in preventing pathogen colonization and enhancing macrophage stimulation.

In order to assess the mixtures, the individual substances and the respective substance combinations were tested together and the results were subsequently compared with one another. In the course of these studies, it could also be demonstrated that the feed and drinking water additives raised the phagocytic activity of the cells more efficiently than the cumulative effects of the individual substances. Thus, the synergistic effect of the components contained in the feed and drinking water additives could be proved in this test system, too.

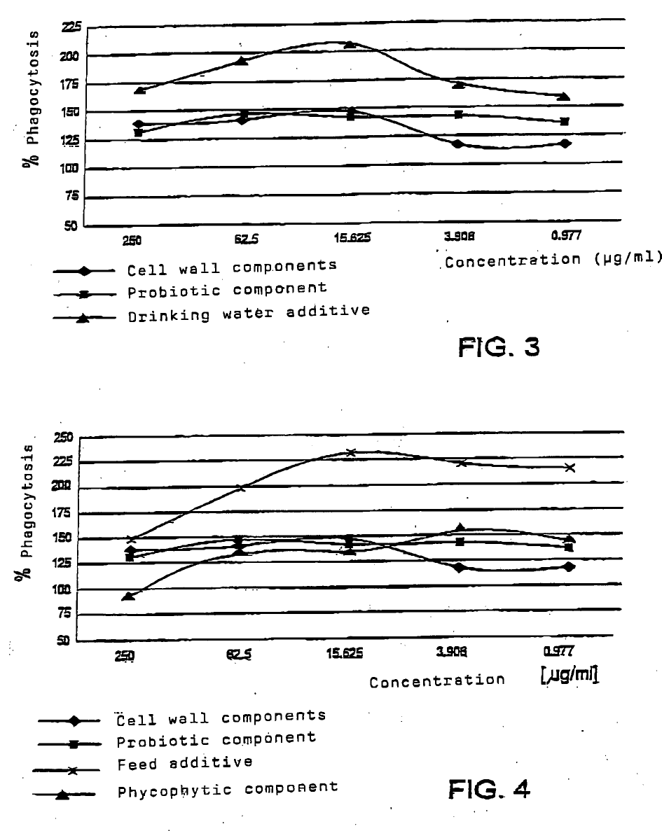

The concentration ranges required for the use of the individual substances with a view to obtaining the desired synergistic effects according to the invention were determined in in vitro experiments using a cell culture test system. In doing so, different substances such as, for instance, Streptococcus faecium IMB 52 (DSM 3530), Bacillus subtilis cell walls, probiotic components and phycophytic components were used in the test system at the concentrations indicated in FIGS. 3 and 4, and the indicated results were obtained. These findings were subsequently translated into in vivo feeding experiments with the following results having been obtained while maintaining the indicated ratios: Thus, from the optimum in vitro amount of use of cell wall components ranging between 0.2 and 19.5 μg/ml, an amount of use ranging between 0.2 and 20 g/t results in ready-made feed. This substance amount must be present in one kilogram feed additive, which means that the additive must contain the substances in a 1000-time concentration at a dosage of 1 kg/t ready-made feed. Hence results an amount of 0.2 to 20 g cell wall components per kilogram feed additive. The amounts for the prebiotic and phycophytic components can be calculated accordingly, with an amount of use of these two components raised by a factor 25 to 35 resulting from further investigations into in vivo uses. This factor takes into account the larger amount of microorganisms present in vivo in the gut per gram or milliliter as compared to the in vitro test system. Hence result the indicated amounts for the probiotic and phycophytic components in the feed additive and/or drinking water additive. By contrast, the amount of probiotic components is determined through the number of colony-forming units.

Drinking Water Additive

Values in % (difference results of test substances in % phagocytosis/control)

| | Cell wall | | | | |
|---|---|---|---|---|---|
| | parts | Probiotic | | Drinking | |
| | Bacillus | Component | Σ Com- | water | Synergistic |
| [μg/ml] | subtilis (%) | (%) | ponents | additive | effect |
| 250 | 38.00 | 34.40 | 68.40 | 67.71 | no |
| 62.5 | 41.02 | 46.39 | 87.41 | 90.51 | yes |
| 15.625 | 46.26 | 41.99 | 88.25 | 104.30 | yes |
| 3.906 | 16.60 | 40.60 | 57.20 | 70.40 | yes |
| 0.977 | 15.50 | 33.90 | 49.40 | 61.70 | yes |

Feed Additive

Values in % (difference results of test substances in % phagocytosis/control)

| | | Cell wall | | | | |
|---|---|---|---|---|---|---|
| | Ascophyllum | parts | | | | |
| | nodosum | Bacillus | Probiotic | | | |
| | fine powder | subtilis | Component | Σ | Feed | Synergistic |
| [μg/ml] | (%) | (%) | (%) | Components | additive | effect |
| 250 | −4.92 | 38.00 | 34.40 | 63.48 | 48.18 | no |
| 62.5 | 35.61 | 41.02 | 46.39 | 123.02 | 98.40 | no |
| 15.625 | 37.57 | 46.26 | 41.99 | 125.82 | 130.12 | yes |
| 3.906 | 52.55 | 16.60 | 40.60 | 10.9.75 | 117.61 | yes |
| 0.977 | 41.38 | 15.50 | 33.90 | 90.78 | 112.21 | yes |

The feed additive and/or drinking water additive according to the invention was experimentally tested in chicks by administering to the latter a drinking water additive according to Experiment 1, a feed additive according to Experiment 2, a combination of drinking water additive and feed additive according to Experiment 3, and a combination of feed additive and drinking water additive at a salmonella challenge according to Experiment 4.

Experiment 1

1,400 broiler chicks (50% male, 50% female) were monitored from life days 1 to 42 and divided into groups of 280 animals each. The animals were given drinking water and feed ad lib. The groups of animals which received a drinking water additive were given the same on day 1 by spraying of the drinking water additive, on days 2 and 3 by the application of drinking water.

The division into groups was effected in the following manner: a control group received no drinking water additive at all; group 1 received a drinking water additive comprised of cell wall components of Bacillus subtilis, and prebiotic fructooligosaccharide; group 2 received Streptococcus faecium IMB 52 (DSM 3530) and prebiotic fructooligosaccharide; group 3 received Bacillus subtilis cell wall components, Streptococcus faecium IMB 52 (DSM 3530) and prebiotic fructooligosaccharide; and group 4 received Bacillus subtilis cell wall components plus Streptococcus faecium IMB 52 (DSM 3530) plus prebiotic fructooligosaccharide plus bifidobacteria. The living mass development of the broilers as well as their mortality rates are indicated in the Tables below. It is clearly apparent from these studies that those animals which had received both cell wall components of Bacillus subtilis and Streptococcus faecium IMB 52 (DSM 3530) clearly outdid all the other groups, both in terms of living mass development and in terms of mortality.

| | | | | |
|---|---|---|---|---|
| Control | Group 1 | Group 2 | Group 3 | Group 4 |

Living mass development (g)

| | | | | | |
|---|---|---|---|---|---|
| Day 1 | 45.1 | 45.0 | 44.6 | 44.8 | 45.1 |
| Day 4 | 65.3 | 696.0 | 66.7 | 67.9 | 69.3 |
| Day 7 | 135.7 | 136.4 | 139.4 | 137.2 | 140.4 |
| Day 14 | 306.8 | 310.7 | 318.3 | 320.6 | 328.0 |
| Day 21 | 599.1 | 599.0 | 603.7 | 610.2 | 624.3 |
| Day 35 | 1396.5 | 1406.3 | 1433.4 | 1420.5 | 1434.8 |
| Day 42 | 1865.4 | 1873.6 | 1888.2 | 1892.7 | 1918.4 |

Mortality (%)

| | | | | | |
|---|---|---|---|---|---|
| Day 4 | 0 | 0 | 0 | 0 | 0 |
| Day 7 | 1.14 | 1.14 | 0.98 | 0.86 | 0.57 |
| Day 14 | 2.20 | 2.00 | 2.00 | 2.00 | 1.71 |
| Day 21 | 2.57 | 2.57 | 2.31 | 2.29 | 2.29 |
| Day 35 | 5.14 | 5.18 | 4.57 | 4.00 | 4.00 |
| Day 42 | 6.29 | 6.36 | 6.19 | 6.12 | 6.00 |

Experiment 2

1,250 broiler chicks (50% male, 50% female) were monitored from life days 1 to 42 and divided into groups of 250 animals each. The animals received a starter feed from day 1 till day 21, furthermore they were given drinking water and feed ad lib.

The division into groups was effected in the following manner: a control group received no drinking water additive at all; group 1 received a feed water additive comprised of cell wall components of Bacillus subtilis, and prebiotic fructooligosaccharide; group 2 received Streptococcus faecium IMB 52 (DSM 3530) and prebiotic fructooligosaccharide; group 3 received Bacillus subtilis cell wall components, Streptococcus faecium IMB 52 (DSM 3530) and prebiotic fructooligosaccharide; group 4 received Bacillus subtilis cell wall components plus Streptococcus faecium IMB 52 (DSM 3530) plus prebiotic fructooligosaccharide plus Ascophyllum nodosum; and group 5 received Bacillus subtilis cell wall components plus Streptococcus faecium IMB 52 (DSM 3530) plus prebiotic fructooligosaccharide plus Ascophyllum nodosum plus bifidobacteria. In experiment 2, both the living mass development of the broilers and their mortality as well as feed utilization were investigated. From this experiment, it is clearly apparent that groups 3 to 5, which had received Bacillus subtilis cell wall components and Streptococcus faecium IMB 52 (DSM 3530) as well as other components, clearly outdid all the other groups both in terms of living mass development and in terms of mortality and feed utilization.

| | | | | | |
|---|---|---|---|---|---|
| Control | Group 1 | Group 2 | Group 3 | Group 4 | Group 5 |

Living mass development (g)

| | | | | | | |
|---|---|---|---|---|---|---|
| Day 7 | 134 | 134 | 133 | 136 | 138 | 136 |
| Day 14 | 280 | 278 | 278 | 284 | 285 | 288 |
| Day 21 | 572 | 576 | 574 | 580 | 586 | 591 |
| Day 35 | 1613 | 1630 | 1634 | 1660 | 1663 | 1674 |
| Day 42 | 2121 | 2145 | 2156 | 2179 | 2174 | 2198 |

Mortality (%)

| | | | | | | |
|---|---|---|---|---|---|---|
| Day 7 | 0.86 | 1.14 | 1.14 | 0.57 | 0.64 | 0.49 |
| Day 14 | 2.00 | 2.00 | 2.00 | 1.71 | 1.65 | 1.53 |
| Day 21 | 2.57 | 2.48 | 2.42 | 2.29 | 2.29 | 2.29 |
| Day 35 | 5.14 | 4.57 | 4.63 | 4.00 | 4.14 | 3.52 |
| Day 42 | 6.29 | 6.14 | 6.14 | 6.00 | 6.05 | 5.85 |

Feed utilization (FCR; g/g)

| | | | | | | |
|---|---|---|---|---|---|---|
| Day 7 | 1.42 | 1.41 | 1.39 | 1.38 | 1.38 | 1.35 |
| Day 14 | 1.60 | 1.58 | 1.59 | 1.57 | 1.58 | 1.56 |
| Day 21 | 1.71 | 1.69 | 1.70 | 1.67 | 1.68 | 1.65 |
| Day 35 | 1.78 | 1.76 | 1.73 | 1.72 | 1.70 | 1.71 |
| Day 42 | 1.90 | 1.88 | 1.85 | 1.81 | 1.80 | 1.76 |

Experiment 3

1,750 broiler chicks (50% male, 50% female) were monitored from life days 1 to 49 and divided into groups of 350 animals each. The animals of group 1 received a drinking water additive, group 2 received a feed additive, and group 3 received a combination of drinking water additive and feed additive, furthermore they were given drinking water and feed ad lib.

The division into groups was effected in the following manner: a control group received no feed or drinking water additive at all; group 2 received Streptococcus faecium IMB 52 (DSM 3530) and prebiotic fructooligosaccharide; and group 3 received Bacillus subtilis cell wall components, Streptococcus faecium IMB 52 (DSM 3530) and prebiotic fructooligosaccharide. From this, it is clearly apparent that the group of animals which had received both feed additives and drinking water additives, clearly outdid all the other groups both in terms of living mass development and in terms of mortality, this superiority exceeding the sum of the effects of the individual components.

| | | | |
|---|---|---|---|
| Control | Group 1 | Group 2 | Group 3 |

Living weight (g)

| | | | | |
|---|---|---|---|---|
| Day 7 | 130.7 | 139.4 | 137.2 | 140.4 |
| Day 14 | 407.4 | 414.2 | 417.4 | 422.8 |
| Day 21 | 838.6 | 849.8 | 855.6 | 862.5 |
| Day 35 | 1698.4 | 1719.2 | 1715.4 | 1711.8 |
| Day 49 | 2190.6 | 2183.8 | 2203.6 | 2295.5 |

Mortality (%)

| | | | | |
|---|---|---|---|---|
| Day 7 | 1.60 | 0.66 | 0.66 | 0.40 |
| Day 14 | 2.00 | 1.66 | 1.66 | 1.20 |
| Day 21 | 4.33 | 2.80 | 2.88 | 2.40 |
| Day 35 | 5.60 | 4.90 | 3.77 | 30.3 |
| Day 49 | 6.80 | 6.10 | 5.90 | 4.80 |

Experiment 4

450 broiler chicks (50% male, 50% female) were monitored from life days 1 to 49 and divided into groups of 90 animals each. On day 1 the animals were administered a drinking water additive by spraying, on days 2 and 3 they received a drinking water additive via drinking water and a feed additive via the starter feed for a period of 21 days. 24 hours after the administration of the drinking water additive, the animals were inoculated with 0.25 ml Salmonella enteridis (4×10⁴ cfu/ml).

The division into groups was effected in the following manner: a control group (+) received no feed or drinking water additive at all, yet was inoculated; group 1 received Bacillus subtilis cell wall components plus Streptococcus faecium IMB 52 (DSM 3530) plus prebiotic fructooligosaccharide plus bifidobacteria; group 2 received Bacillus subtilis cell wall components plus Streptococcus faecium IMB 52 (DSM 3530) plus prebiotic fructooligosaccharide plus Ascophyllum nodosum plus bifidobacteria; group 3 received a combination of drinking water additive and feed additive; and a control group (−) received no feed and drinking water additives at all, and were not inoculated either. From this experiment, it is clearly apparent that the infection of organs with Salmonella enteritidis could be markedly lowered in animals which had received a feed additive and/or drinking water additive according to the invention as against the control group, which had not received any feed additive and/or drinking water additive, wherein those animals which had received both feed and drinking water additives showed no organ infection any longer at the end of the addition. To sum up, it should be noted that it is feasible, by the aid of the feed additive and/or drinking water additive according to the invention, to not only significantly raise the living mass weights of animals and lower their mortality, but also suppress almost completely organ infections with pathogenic germs.

| | Control (+) | Group 1 | Group 2 | Group 3 | Control (−) |
|---|---|---|---|---|---|
| Day 7 | 10/15 | 3/15* | 3/15* | 1/15* | 0/15 |
| Day 14 | 9/15 | 2/15* | 2/15* | 1/15* | 0/15 |
| Day 21 | 8/15 | 2/15* | 2/15* | 0/15* | 0/15 |
| Day 35 | 7/15 | 0/15* | 1/15* | 0/15* | 0/15 |
| Day 49 | 5/15 | 0/15* | 0/15* | 0/15* | 0/15 |

*significant difference (P < 0.05)

1. A feed additive and/or drinking water additive for domestic animals, which contains at least bacteria of the strain Streptococcus faecium IMB 52 (DSM 3530), wherein standardized cell wall components from Bacillus sp. and/or Streptoccocus sp. and/or Bifidobacterium sp. as well as inulin are additionally contained.

2. A feed additive and/or drinking water additive according to claim 1, wherein the standardized cell wall components are selected from the strain Bacillus subtilis.

3. A feed additive and/or drinking water additive according to claim 1, wherein the standardized cell wall components from Bacillus subtilis are contained in an amount of from 0.2 to 40 g/kg feed additive and/or drinking water additive and, in particular, 5 to 15 g/kg.

4. A feed additive and/or drinking water additive according to claim 1, wherein inulin is isolated from leek, onion, garlic, artichoke, wheat, chicory, topinambour, tomato, banana and/or rye and contained in an amount of from 100 to 950 g/kg feed additive and/or drinking water additive.

5. A feed additive and/or drinking water additive according to claim 4, wherein said inulin is contained in the feed additive in an amount of from 100 to 700 g/kg, particularly 320 to 540 g/kg.

6. A feed additive and/or drinking water additive according to claim 4, wherein said inulin is contained in the drinking water additive in an amount of from 530 to 950 g/kg, particularly 820 to 920 g/kg.

7. A feed additive and/or drinking water additive according to claim 1, wherein the bacteria of the strain Streptococcus faecium IMB 52 (DSM 3530) are partially replaced with at least one further bacterial strain.

8. A feed additive and/or drinking water additive according to claim 1 any one of claims 1 to 7, wherein 10 to 50% of the bacteria of the strain Streptococcus faecium IMB 52 (DSM 3530) are replaced with at least one further bacterial strain.

9. A feed additive and/or drinking water additive according to claim 1, wherein the at least one further bacterial strain is selected from the strains Bifidobacteruim thermophilum (I-01) (DSM 14411), Bifidobacterium thermophilum (I-07) (DSM 14412), Bifidobacteruim boum (I-12) (DSM 14413), Bifidobacterium thermophilum (I-15) (DSM 14414), Bifidobacteruim thermophilum (I-19) (DSM 14415) und Bifidobacterium thermophilum (I-20) (DSM 14416).

10. A feed additive and/or drinking water additive according to claim 1, wherein brown marine algae are additionally contained.

11. A feed additive and/or drinking water additive according to claim 9, wherein said brown algae are comprised of Ascophyllum nodosum.

12. A feed additive and/or drinking water additive according to claim 10, wherein Ascophyllum nodosum is contained in an amount of from 300 to 800 g/kg, particularly 450 to 550 g/kg, feed additive.

13. A feed additive and/or drinking water additive according to claim 1, wherein 8 g Bacillus subtilis, 60.0 g Streptococcus faecium IMB 52 (DSM 3530), 432 g Bifidobacterium thermophilum, balance Ascophyllum nodosum, are contained per kg feed additive and/or drinking water additive.

14. A feed additive and/or drinking water additive according to claim 1, wherein 10 g Bacillus subtilis, 24.75 Streptococcus faecium IMB 52 (DSM 3530), 10.5 g Bifidobacterium thermophilum (DSM 14414), balance inulin, particularly from chicory, are contained per kg feed additive and/or drinking water additive.
